(19) United States
(12) Patent Application Publication
(10) Pub. No.: US 2021/0166579 A1
(43) Pub. Date: Jun. 3, 2021

(54) HEALTH MANAGEMENT DEVICE, HEALTH MANAGEMENT METHOD, AND NON-TRANSITORY RECORDING MEDIUM STORING PROGRAM

(71) Applicant: OMRON HEALTHCARE Co., Ltd., Kyoto (JP)

(72) Inventors: Tomohiro KUKITA, Kyoto (JP); Masahiko YUMOTO, Kyoto (JP); Tamio UEDA, Kyoto (JP)

(21) Appl. No.: 17/173,927

(22) Filed: Feb. 11, 2021

Related U.S. Application Data
(63) Continuation of application No. PCT/JP2019/029022, filed on Jul. 24, 2019.

(30) Foreign Application Priority Data
Aug. 20, 2018 (JP) 2018-154192

(51) Int. Cl. G09B 19/00; G16H 20/00; G09B 7/06

(52) U.S. Cl. CPC G09B 19/00; G09B 7/06; G16H 20/00

(57) ABSTRACT

A health management device according to an aspect of the present invention includes an inquiry unit configured to inquire a user as to a reason why the user could not perform a behavior in a second time period in a manner that satisfies a condition, in a case in which it is determined that an indicator has not changed in a first time period toward achieving a predetermined goal and it is determined that the user has not performed the behavior in the second time period, which includes a portion of the first time period, in a manner that satisfies a predetermined condition, and an information presentation unit configured to present, on the basis of an answer input by the user, information that facilitates achieving the goal, to the user.

### CROSS-REFERENCE TO RELATED APPLICATIONS

This application is the U.S. national stage application filed pursuant to 35 U.S.C. 365(c) and 120 as a continuation of International Patent Application No. PCT/JP2019/029022, filed Jul. 24, 2019, which application claims priority from Japanese Patent Application No. 2018-154192, filed Aug. 20, 2018, which applications are incorporated herein by reference in their entireties.

### TECHNICAL FIELD

An aspect of the present invention relates to technology for managing the health of a user.

### BACKGROUND ART

A known system provides advice to a user to help them achieve set goals relating to the health of the user. For example, in Patent Document 1, an advice generation system is described that determines motivation for achieving a goal on the basis of achievement state information indicative of the achievement state of the goal to be achieved by a user, and action amount information indicative of the cumulative amount of action taken by the user to achieve the goal, and generates advice for the user on the basis of the determination result of the motivation.

### CITATION LIST

### Patent Literature

Patent Document 1: JP 2016-106698 A

### SUMMARY OF INVENTION

### Technical Problem

In the advice generation system described in Patent Document 1, when the action amount is low, advice is provided requesting to increase the action amount. However, in the case in which there are circumstances preventing the user from performing behavior, this may reduce the motivation of the user, who wants to perform a behavior to achieve a goal. Thus, the advice generation system may not be able to provide appropriate advice.

The present invention has been made in light of the circumstances described above, and an object of the present invention is to provide a health management device, a health management method, and a non-transitory recording medium storing a program capable of presenting appropriate advice that facilitates achieving a goal relating to health.

### Solution to Problem

The present invention adopts the following configurations in order to solve the above problems.

A health management device according to an aspect includes, a measurement data acquisition unit configured to acquire measurement data output from a measurement device configured to measure an indicator relating to a user, a first determination unit configured to determine, on the basis of the measurement data, whether or not the indicator has changed in a first time period toward achieving a preset goal, a behavior data acquisition unit configured to acquire behavior data relating to behavior of the user, a second determination unit configured to determine, on the basis of the behavior data, whether or not the user has performed the behavior in a second time period in a manner that satisfies a preset condition, the second time period including at least a portion of the first time period, an inquiry unit configured to inquire the user as to a reason why the user could not perform the behavior in the second time period in a manner that satisfies the condition, in a case in which the first determination unit determines that the indicator has not changed in the first time period toward achieving the goal and the second determination unit determines that the user has not performed the behavior in the second time period in a manner that satisfies the condition, and an information presentation unit configured to present, on the basis of an answer input by the user to an inquiry from the inquiry unit, information that facilitates achieving the goal, to the user and to determine, in a case in which the answer includes a reason why the behavior could not be performed, whether or not to accept the reason included in the answer, and present different information to the user for a case in which the reason included in the answer is tolerant and for a case in which the reason included in the answer is not tolerant.

According to the configuration described above, an inquiry is presented to the user as to the reason why the user could not perform the behavior in the second time period in a manner that satisfies the condition, when it is determined that the indicator has not changed in the first time period toward achieving the goal and it is determined that the user has not performed the behavior in the second time period in a manner that satisfies the condition, and information that facilitates achieving the goal is presented to the user on the basis of the answer input by the user. Accordingly, for example, a message can be presented to the user prompting to exercise as much as they are able to when the user answers with a reason as to why they could not perform the behavior, and a message can be presented to the user strongly prompting to exercise when the user answers with no reason as to why they could not perform the behavior. Accordingly, advice can be presented to the user without reducing the motivation of the user. In other words, appropriate advice that facilitates achieving a goal relating to the health of the user can be provided to the user.

According to this configuration, the information presentation unit may be configured to determine, in a case in which the answer includes a reason why the behavior could not be performed, whether or not to accept the reason included in the answer, and present different information to the user for a case in which the reason included in the answer is tolerant and for a case in which the reason included in the answer is not tolerant.

According to the configuration described above, whether or not the reason input by the user is reasonable is determined. Accordingly, a message can be presented to the user prompting to exercise as much as they are able to when circumstances that prevented from performing the behavior belongs to the user, and a message can be presented to the user strongly prompting to exercise when there are no circumstances that prevented from performing the behavior.

In an aspect, provided is a measurement data acquisition unit configured to acquire measurement data output from a measurement device configured to measure an indicator relating to a user, a first determination unit configured to determine, on the basis of the measurement data, whether or not the indicator has changed in a first time period toward achieving a preset goal, a behavior data acquisition unit configured to acquire behavior data relating to behavior of the user; a second determination unit configured to determine, on the basis of the behavior data, whether or not the user has performed the behavior in a second time period in a manner that satisfies a preset condition, the second time period including at least a portion of the first time period, an inquiry unit configured to inquire the user as to a reason why the user could not perform the behavior in the second time period in a manner that satisfies the condition, in a case in which the first determination unit determines that the indicator has not changed in the first time period toward achieving the goal and the second determination unit determines that the user has not performed the behavior in the second time period in a manner that satisfies the condition, and an information presentation unit configured to present, on the basis of an answer input by the user to an inquiry from the inquiry unit, information that facilitates achieving the goal, to the user, wherein the goal may include a target value, and the first determination unit is configured to determine, on the basis of the measurement data, whether the first time period corresponds to one of a first stage in which the indicator has not achieved the target value or a second stage in which the indicator has achieved the target value, and determine, according to different criteria for the first stage and the second stage, whether or not the indicator has changed in the first time period toward achieving the goal.

According to the configuration described above, health of the user can be managed not only in a stage of improving the health state or a stage of maintaining the health state, but also in both.

In an aspect, the first determination unit is configured, for the first stage, to determine that the indicator has changed in the first time period toward achieving the goal in a case in which the indicator shows sign of a decreasing trend, and determine that, the indicator has not changed in the first time period toward achieving the goal in a case in which the indicator shows no sign of a decreasing trend, and for the second stage, to determine that the indicator has changed in the first time period toward achieving the goal in a case in which an average value of the indicator for the first time period is less than the target value, and determine that the indicator has not changed in the first time period toward achieving the goal in a case in which the average value of the indicator for the first time period is greater than the target value.

According to the configuration described above, when the goal is to reduce an indicator, such as weight or blood pressure, whether or not the indicator has changed in the first time period toward achieving the goal can be determined.

In an aspect, the second time period may include at least a portion of the first time period and a day prior to the first time period.

The indicator in the first time period is affected by the behavior on the day prior to the first time period. Thus, according to the configuration described above, whether or not the cause of the indicator not changing in the first time period toward achieving the goal is the behavior can be accurately measured.

### Advantageous Effects of Invention

According to the present invention, a health management device, a health management method, and a non-transitory recording medium storing a program capable of presenting appropriate advice that facilitates achieving a goal relating to health.

### BRIEF DESCRIPTION OF DRAWINGS

FIG. 1 is a diagram illustrating a health management system according to an embodiment.

FIG. 2 is a block diagram illustrating a hardware configuration of a terminal device according to an embodiment.

FIG. 3 is a block diagram illustrating a software configuration of the terminal device illustrated in FIG. 2.

FIG. 4 is a flowchart illustrating a health management method executed by the terminal device illustrated in FIG. 2.

### DESCRIPTION OF EMBODIMENTS

Hereinafter, an embodiment of the present invention will be described with reference to the drawings.

### Application Example

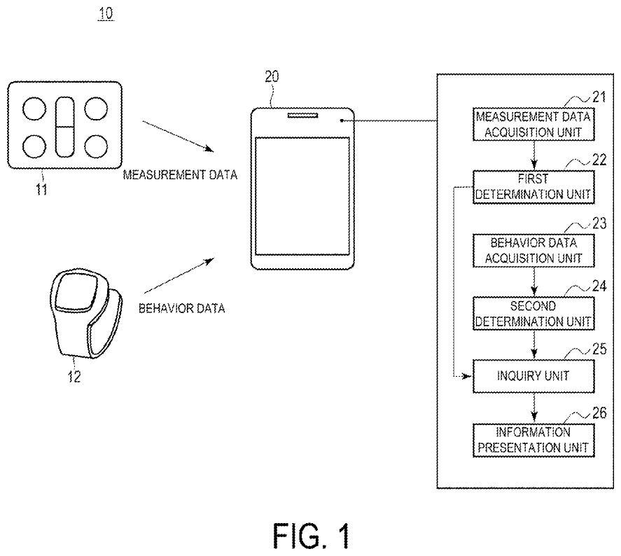

With reference to FIG. 1, an example of a case to which the present invention is applied will be described. FIG. 1 illustrates a health management system 10 according to an embodiment. In the example of FIG. 1, the health management system 10 includes a weight scale 11, an exercise amount meter 12, and a terminal device 20. The weight scale 11 and the exercise amount meter 12 are used by a user who owns the terminal device 20.

The weight scale 11 measures the weight of the user and generates measurement data that includes a measurement value of the weight. Weight is an example of an indicator relating to a user, and the weight scale 11 is an example of a measuring device that measures an indicator relating to a user. In the example of FIG. 1, the weight scale 11 is worn on the user's arm.

The exercise amount meter 12 measures the amount of exercise of the user and generates measurement data that includes a measurement value of the amount of exercise. The amount of exercise measured by the exercise amount meter 12 includes not only exercise relating to athletic activities such as sports, but also the amount of exercise (also referred to as amount of activity) relating to life activities, such as walking and housework. The amount of exercise can be expressed, for example, in calorie consumption. The amount of exercise is an example of an indicator relating to the behavior of a user, and the exercise amount meter 12 is an example of a measuring device that measures an indicator relating to the behavior of a user.

The terminal device 20 may be a computer such as a smart phone, a tablet personal computer (PC), a mobile phone, a laptop PC, and a desktop PC, for example. The terminal device 20 includes a measurement data acquisition unit 21, a first determination unit 22, a behavior data acquisition unit 23, a second determination unit 24, an inquiry unit 25, and an information presentation unit 26. The terminal device 20 is configured to manage the health of the user in terms of weight and corresponds to the “health management device” of the present invention.

The measurement data acquisition unit 21 acquires measurement data output from the weight scale 11. In one example, the measurement data is displayed on the weight scale 11 and the user inputs the measurement data into the terminal device 20. In another example, the terminal device 20 can wirelessly communicate with the weight scale 11 and receives the measurement data from the weight scale 11. For example, the user measures their weight with the weight scale 11 after waking up each day, so that the weight (measurement values) of each morning are recorded in the terminal device 20.

The first determination unit 22 determines, on the basis of the measurement data acquired by the measurement data acquisition unit 21, whether or not the weight of the user has changed in a first time period toward achieving a preset goal. For example, the user's goal may be to reduce weight to a target value. The target value may be a value specified by the user him or herself or may be a value specified by another person such as a physician. For example, the first determination unit 22 determines a trend in the change of the weight on the basis of the weight measurement values for each day in the first time period. In the case in which the weight shows sign of a decreasing trend, the first determination unit 22 determines that the weight has changed in the first time period toward achieving the goal and, otherwise, the first determination unit 22 determines that the weight has not changed in the first time period toward achieving the goal.

The behavior data acquisition unit 23 acquires measurement data output from the exercise amount meter 12 as behavior data relating to the behavior of the user. For example, the terminal device 20 can wirelessly communicate with the exercise amount meter 12 and receives the measurement data from the exercise amount meter 12. The daily amount of exercise (measurement value) is recorded as behavior data in the terminal device 20.

The second determination unit 24 determines, on the basis of the behavior data acquired by the behavior data acquisition unit 23, whether or not exercise has been performed in a second time period in a manner that satisfies a preset condition. The second time period includes at least a portion of the first time period. In this example, the second time period is the same time period as the first time period. For example, the second determination unit 24 determines that the user has performed exercise in the second time period in a manner that satisfies the condition in the case in which the total amount of exercise for the second time period is greater than a preset threshold and, otherwise, determines that the user has not performed exercise in the second time period in a manner that satisfies the condition.

The inquiry unit 25 asks the user as to the reason why the user could not perform exercise in the second time period in a manner that satisfies the condition, when the first determination unit 22 determines that the weight has not changed in the first time period toward achieving the goal and the second determination unit 24 determines that the user has not performed exercise in the second time period in a manner that satisfies the condition. For example, the inquiry unit 25 displays on a display device an inquiry screen that includes a message asking for the reason why exercise could not be performed and a plurality of options that are candidates for the answer. For example, one of the options may be the answer that work was busy, and another one of the options may be the answer that there is no special reason.

The information presentation unit 26 presents, on the basis of the answer to the inquiry performed by the inquiry unit 25 input by the user, the user with information (hereinafter, also referred to as advice) that facilitates achieving the goal. When the user answered as to a reason (an excuse) why they could not perform exercise, for example, the user answered a reason that the work was busy, the information presentation unit 26 presents to the user a message prompting to exercise as much as they are able to. On the other hand, when the user answered that there is no special reason, the information presentation unit 26 presents a message to the user that strongly prompts to exercise.

In addition, when the user answered a reason why they could not perform the behavior, the information presentation unit 26 determines whether or not the reason included in the answer input by the user is tolerant. The advice presented to the user may differ depending on whether the reason is tolerant or not tolerant. Tolerating the reason corresponds to acknowledging that there were circumstances that prevented the user from performing the behavior (in this example, exercise).

In the case in which there are circumstances preventing the user from exercising, by presenting the user with a message strongly prompting to exercise, the motivation of the user, who wants to exercise to achieve a goal, may be reduced. The terminal device 20 with the configuration described above presents to the user a message prompting to exercise as much as they are able to when the reason input by the user is tolerant, and presents to the user a message strongly prompting to exercise when the reason input by the user is not tolerant or the user answered that there is no reason. Accordingly, advice can be presented to the user without reducing the motivation of the user. In other words, appropriate advice that facilitates achieving a goal relating to the health of the user can be provided to the user.

Next, a health management device according to an embodiment will be described in further detail.

### Configuration Example

### Hardware Configuration

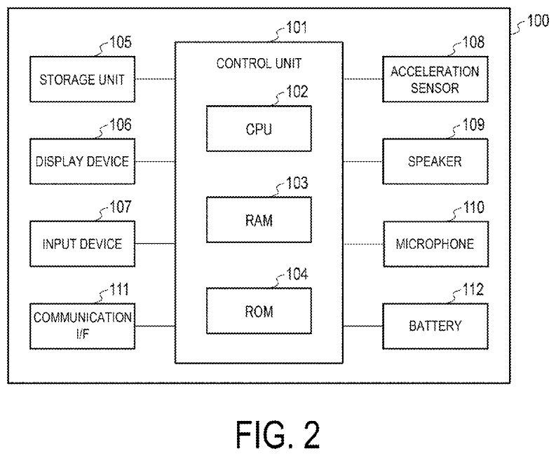

An example of the hardware configuration of a terminal device 100, i.e., a health management device according to an embodiment will be described with reference to FIG. 2. FIG. 2 illustrates an example of the hardware configuration of the terminal device 100. In the example of FIG. 2, the terminal device 100 is a mobile device such as a smart phone and includes a control unit 101, a storage unit 105, a display device 106, an input device 107, an acceleration sensor 108, a speaker 109, a microphone 110, a communication interface 111, and a battery 112.

The control unit 101 includes a central processing unit (CPU) 102, a random access memory (RAM) 103, a read only memory (ROM) 104, and the like and controls each component. For example, the storage unit 105 is an auxiliary storage device, such as a hard disk drive (HDD), and a semiconductor memory (for example, a flash memory), and stores, in a non-volatile manner, programs executed by the control unit 101, setting data necessary for executing the programs, measurement data, and the like. A storage medium included in the storage unit 105 is a medium that accumulates information such as the program by electrical, magnetic, optical, mechanical, or chemical action so that a computer, a machine, or the like can read recorded information such as the program. Note that at least one or all of the programs may be stored in the ROM 104.

The display device 106 is controlled by the control unit 101 and displays information such as advice. The display device 106 may be an organic electroluminescence (EL) display, for example. Organic EL displays are sometimes referred to as organic light emitting diode (OLED) displays. Also, the display device 106 may be a display device of a different type, such as a liquid crystal display (LCD) device, for example.

The input device 107 allows the user to input an instruction to the terminal device 100. The input device 107 accepts an operation by a user, for example, an operation of inputting a measurement value of weight. The input device 107 typically includes a touch panel provided on the screen of the display device 106. Instead of or in addition to the touch panel, the input device 107 may include one or more hardware buttons.

The acceleration sensor 108 is, for example, a triaxial acceleration sensor and outputs an acceleration signal representing acceleration in three directions orthogonal to each other. The speaker 109 is supplied with an audio signal from the control unit 101 and converts the audio signal into sound. The microphone 110 converts the sound into an electrical signal and provides the electrical signal to the control unit 101. The microphone 110 allows the user to operate the terminal device 100 by voice.

The communication interface 111 is an interface for communicating with an external communication device. The communication interface 111 includes, for example, a short-range wireless communication module, such as a Bluetooth (trade name) module or a Bluetooth Low Energy (BLE) module and communicates directly with the communication device (for example, a measurement device). The communication interface 111 may include a wireless module such as a wireless local area network (LAN) module instead of or in addition to the short-range wireless communication module. The communication interface 111 communicates with a communication device (for example, a server) via a communication network such as the Internet using a Bluetooth module or a wireless LAN module. The communication interface 111 may also include a terminal such as a Universal Serial Bus (USB) connector to communicate with the communication device in a wired manner.

The battery 112 supplies power to each component in the terminal device 100. The battery 112 is a rechargeable battery, for example.

Note that, with regard to the specific hardware configuration of the terminal device 100, a component can be omitted, replaced, and added as appropriate according to the embodiment. For example, the control unit 101 may include a plurality of processors.

### Software Configuration

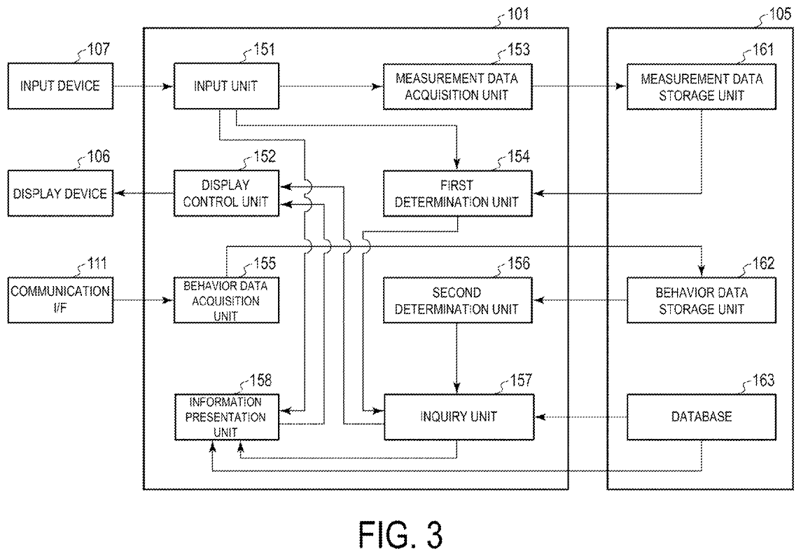

An example of the software configuration of the terminal device 100 will be described with reference to FIG. 3. FIG. 3 illustrates an example of the software configuration of the terminal device 100. In the example of FIG. 3, the terminal device 100 includes an input unit 151, a display control unit 152, a measurement data acquisition unit 153, a first determination unit 154, a behavior data acquisition unit 155, a second determination unit 156, an inquiry unit 157, an information presentation unit 158, a measurement data storage unit 161, a behavior data storage unit 162, and a database 163. The input unit 151, the display control unit 152, the measurement data acquisition unit 153, the first determination unit 154, the behavior data acquisition unit 155, the second determination unit 156, the inquiry unit 157, and the information presentation unit 158 executes the following processing by the control unit 101 of the terminal device 100 executing a program stored in the storage unit 105. When the control unit 101 executes the program, the control unit 101 deploys the program in the RAM 103. Then, the control unit 101 causes the CPU 102 to interpret and execute the program deployed in the RAM 103 to control each component. The measurement data storage unit 161, the behavior data storage unit 162, and the database 163 are provided in the storage unit 105.

The input unit 151 accepts input from the user. The input unit 151 receives, from the input device 107, an operation signal corresponding to the operation which the user performed on the input device 107, and determines, on the basis of the operation signal, the contents of the instruction input by the user. For example, when the user uses the input device 107 to input goal information, which is information relating to a goal, the input unit 151 sends the input goal information to the first determination unit 154. Accordingly, the goal of the user is set at the first determination unit 154. Additionally, when the user uses the input device 107 to input the weight measurement value, the input unit 151 provides information representing the weight measurement value to the measurement data acquiring unit 153. When the user uses the input device 107 to enter an answer to the inquiry screen described below, the input unit 151 provides information representing the content of the answer to the information presentation unit 158.

The display control unit 152 controls the display device 106 and causes information to be displayed on the display device 106. For example, the display control unit 152 causes the display device 106 to display a screen for setting a goal. The display control unit 152 causes the display device 106 to display the inquiry screen for inputting the reason why exercise could not be performed. The display control unit 152 receives advice to be presented to the user from the information presentation unit 158 and causes the display device 106 to display this advice.

The measurement data acquisition unit 153 acquires measurement data generated by the weight scale (for example, the weight scale 11 illustrated in FIG. 1) and stores the measurement data in the measurement data storage unit 161. In the example of FIG. 3, the user inputs measurement data using the input device 107, and the measurement data acquiring unit 153 acquires the measurement data input by the user via the input unit 151. In another example, the measurement data acquisition unit 153 receives measurement data from the weight scale via the communication interface 111. The user uses the weight scale to measure their weight every day, and thus the measurement data of each day is accumulated in the measurement data storage unit 161.

The first determination unit 154 reads the measurement data from the measurement data storage unit 161 and determines, on the basis of the measurement data, whether or not the weight has changed in the first time period toward achieving a predetermined goal. The first determination unit 154 uses measurement values of each day in the first time period to make a determination. For example, when the first time period is from April 1 to April 7, the measurement values of 7 days from April 1 to April 7 is used. When the user takes a plurality of measurements in one day, an average of the plurality of measurement values obtained from the measurements may be used as the measurement value of the day for the determination. Also, a measurement value obtained from a measurement performed in a specific time period of each day (for example, from 7 o'clock to 9 o'clock) may be used for determination.

The user's goal is, for example, to make weight below a target value (for example, 60 (kg)). Making weight below a target value can include reducing weight to the target value as well as keeping the weight below the target value. In this case, the user's goal can be divided into a stage of reducing weight below the target value (hereinafter referred to as the first stage) and a stage of keeping weight below the target value (hereinafter referred to as the second stage). The first stage is the stage in which the weight has not reached the target value, and the second stage is the stage in which the weight has reached the goal. The first determination unit 154 determines whether the first time period corresponds to either one of the first stage and the second stage on the basis of the measurement data. For example, the first determination unit 154 calculates an average value for the weight over the specific time period and determines the stage on the basis of the calculated weight average value. In an example in which the specific time period is a time period just prior to the first time period, for example, in the case in which the calculated weight average value exceeds the target value, the first time period corresponds to the first stage, otherwise the first time period corresponds to the second stage. The specific time period may be the same as the first time period. The specific time period may be the same length as the first time period or may be a different length to the first time period. As an example, when the first time period is from April 1 to April 7, the specific time period may be from March 25 to March 31. The first determination unit 154 makes a determination using different criteria depending on the stage.

In the first stage, the first determination unit 154 determines whether or not weight has a decreasing trend in the first time period. For example, the first determination unit 154 calculates a weight lowering rate from the measurement values of each of the days in the first time period and, in the case in which the weight lowering rate is greater than a preset threshold, the first determination unit 154 determines that the weight has a decreasing trend in the first time period, and otherwise determines that the weight does not have a decreasing trend in the first time period. The weight lowering rate represents, for example, how much weight decreased per day. The weight lowering rate having a greater value indicates a decrease in weight. The weight lowering rate can be determined, for example, by calculating a linear function that fits the measurement value using the least squares method. The threshold is, for example, 0.05 (kg/1 day).

In the second stage, the first determination unit 154 makes a determination on the basis of the average weight value of the first time period, for example. Specifically, the first determination unit 154 calculates the average weight value of the first time period on the basis of the measurement data. In the case in which the average weight value is less than the target value, the first determination unit 154 determines that the weight has changed in the first time period toward achieving the predetermined goal, and otherwise, the first determination unit 154 determines that the weight has not changed in the first time period toward achieving the predetermined goal.

The behavior data acquisition unit 155 acquires behavior data relating to the behavior of the user and stores the acquired behavior data in the behavior data storage unit 162. The behavior data acquisition unit 155 receives measurement data including a measurement value of the amount of exercise from an exercise amount meter (for example, the exercise amount meter 12 illustrated in FIG. 1) via the communication interface 111. The amount of exercise per day is recorded in the behavior data storage unit 162.

The second determination unit 156 reads the behavior data from the behavior data storage unit 162 and determines, on the basis of the behavior data, whether or not the user has performed exercise in the second time period in a manner that satisfies a predetermined condition. The second time period may be the same as the first time period. For example, the first time period and the second time period are both from April 1 to April 7. Also, the second time period may include the day prior to the first time period and at least a portion of the first time period. For example, the first time period is from April 1 to April 7, and the second time period is from March 29 to April 6. The condition is, for example, that the total amount of exercise in the second time period is greater than a preset threshold. The second determination unit 156 calculates the total amount of exercise of the second time period on the basis of the behavior data. In the case in which the calculated total amount of exercise is greater than the threshold, the second determination unit 156 determines that the user performed exercise in the second time period in a manner that satisfies the predetermined condition, and otherwise, the second determination unit 156 determines that the user did not perform exercise in the second time period in a manner that satisfies the predetermined condition. The threshold may be a fixed value or may be set to a value suitable for the user. For example, in the case in which a target value and a time period are specified so that the weight is reduced by 5 kg in two months, the threshold may be determined on the basis of the target value and time period.

The inquiry unit 157 asks the user as to the reason why the user could not perform exercise in the second time period in a manner that satisfies the predetermined condition, when the first determination unit 154 determines that the weight has not changed in the first time period toward achieving the predetermined goal and the second determination unit 156 determines that the user has not performed exercise in the second time period in a manner that satisfies the predetermined condition. For example, the inquiry unit 157 displays the inquiry screen that inquires as to the reason why the user could not exercise in the second time period in a manner that satisfies the predetermined condition. The inquiry screen is displayed on display device 106 via the display control unit 152. The inquiry screen, for example, includes a message saying “The amount of exercise in the time period from April 1 to April 7 was insufficient. Please enter the reason why exercise could not be performed in the time period”.

The information presentation unit 158 presents, on the basis of the answer to the inquiry performed by the inquiry unit 157 input by the user, the user with information that facilitates achieving the predetermined goal. The information presentation unit 158 receives an answer input by the user in response to the inquiry screen. The inquiry screen may further include a plurality of options that are candidates for the answer. For example, the inquiry screen includes an option corresponding to an answer that work was busy, an option corresponding to an answer that there were many days with bad weather, and an option corresponding to an answer that there is no special reason. The user uses the input device 107 to select one of these options, and the information presentation unit 158 receives an answer corresponding to the option selected by the user as the user answer.

Note that the user may use the input device 107 to input characters as an answer. In this case, the information presentation unit 158 analyzes the character string input by the user and selects one of the answers prepared in advance as the answer to the user on the basis of the analysis result. The microphone 110 may be used by the user to input the answer by voice. In this case, the information presentation unit 158 recognizes the voice input by the user, generates a character string, analyzes the generated character string, and selects one of the answers prepared in advance as the answer to the user on the basis of the analysis result.

The information presentation unit 158 presents the user with advice on the basis of the answer input by the user. The information presentation 158 presents the advice to the user on the basis of the presence or absence of a reason why the behavior could not be performed and the content of why the behavior could not be performed in the case of there being a reason. When the user answers with a reason why they could not perform the behavior, the information presentation unit 158 determines whether or not the reason is tolerant and presents to the user information that differs depending on whether the reason is tolerant or not tolerant. When the answer input by the user is that work was busy, the information presentation unit 158 accepts the reason input by the user. When the user inputs an answer that there were many days with bad weather, the information presentation unit 158 acquires weather information showing the weather in the second time period from a server via the communication interface 111. In the case in which there were many rainy days in the second time period, the reason input by the user is accepted. In the case in which there were few rainy days in the second time period, the reason input by the user is not accepted.

The information presentation unit 158 presents advice prompting to exercise with a soft expression in the case in which the reason input by the user is tolerant. For example, the information presentation unit 158 displays on the display device 106 a message saying “Please keep in mind to exercise as much as you are able to. When you can't exercise, please take extra care not to eat as much as usual”. On the other hand, the information presentation unit 158 presents advice prompting to exercise with a stern expression in the case in which the reason input by the user is not tolerant or the user answered that there is no special reason. For example, the information presentation unit 158 displays a message on the display device 106 saying “You are not getting enough exercise. Be sure to exercise everyday to reduce your weight”. These messages are stored in the database 163, and the information presentation unit 158 retrieves the message to be presented to the user from the database 163. Note that the database 163 may be provided on the outside of the terminal device 100, for example, in a server on a communication network. Also, the information presentation unit 158 may present different advice in the case in which the reason input by the user is not tolerant and the case in which the user answered that there is no special reason.

The information presentation unit 158 may present advice to the user when the first determination unit 154 determines that the weight has not changed in the first time period toward achieving the predetermined goal and the second determination unit 156 determines that the user has exercised in the second time period in a manner that satisfies the predetermined condition. When weight is not reduced despite the user performing sufficient exercise, not reducing weight may plausibly have been caused by a behavior other than exercise. Not reducing weight may have been caused by taking too much food intake, for example. Thus, the information presentation unit 158 may present information relating to food intake. For example, the information presentation unit 158 displays on the display device 106 a message saying “You are sufficiently exercising, but your weight is not reduced. Please take care to reduce the amount of food intake”.

The present embodiment is described using an example in which all the functions of the terminal device 100 are implemented by a general purpose processor. However, some or all of the functions may be implemented by one or more dedicated processors.

### Operation Example

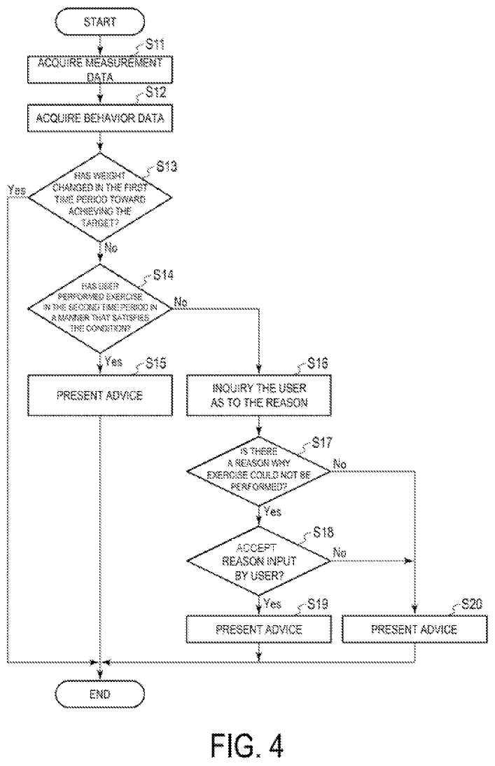

FIG. 4 illustrates an example of an operation flow when the terminal device 20 executes user health management.

In step S11 of FIG. 4, the control unit 101 of the terminal device 100 operates as the measurement data acquisition unit 153 and acquires measurement data from the weight scale. For example, the control unit 101 acquires a measurement value for weight input by the user using the input device 107.

In step S12, the control unit 101 operates as the behavior data acquisition unit 155 and acquires behavior data relating to the exercise of the user. Specifically, for example, the control unit 101 acquires measurement data from the exercise amount meter via the communication interface 111 as behavior data.

The processing in steps S11 and S12 are repeated. Accordingly, measurement data and behavior data are accumulated in the storage unit 105.

Step S13 and the subsequent processing sequences are executed at a predetermined timing. For example, the processing sequence is executed periodically (for example, weekly). Note that the processing sequence may be executed at a timing specified by the user.

In step S13, the control unit 101 operates as the first determination unit 154 and determines, on the basis of the measurement data, whether or not the weight has changed in the first time period toward achieving a predetermined goal. In the case in which the control unit 101 determines that the weight has changed in the first time period toward achieving the target, the processing ends. In the case in which the control unit 101 determines that the weight has not changed in the first time period toward achieving the target, the processing proceeds to step S14. For example, the control unit 101 calculates the average value of the weight for the first time period. In the case in which the calculated average value of the weight for the first time period is less than a threshold, the control unit 101 determines that the weight has changed in the first time period toward achieving the goal, and otherwise, the control unit 101 determines that the weight has not changed in the first time period toward achieving the goal.

In step S14, the control unit 101 operates as the second determination unit 156 and determines, on the basis of the behavior data, whether or not the user performed exercise in the second time period in a manner the satisfies the condition. In the case in which the control unit 101 determines that the user has performed exercise in the second time period in a manner that satisfies the condition, the processing proceeds to step S15, and in the case in which the control unit 101 determines that the user has not performed exercise in the second time period in a manner that satisfies the condition, the processing proceeds to step S16. For example, the control unit 101 calculates the total amount of exercise of the second time period. In the case in which the total amount of exercise is greater than the threshold, the control unit 101 determines that the user has performed exercise in the second time period in a manner that satisfies the condition, and otherwise, the control unit 101 determines that the user did not perform exercise in the second time period in a manner that satisfies the condition.

In step S15, the control unit 101 operates as the information presentation unit 158 and presents advice that facilitates achieving the goal, to the user. For example, the control unit 101 displays on the display device 106 a message saying “You are sufficiently exercising, but your weight is not reduced. Please take care to reduce the amount of food intake”.

In step S16, the control unit 101 operates as the inquiry unit 157 to ask the user as to the reason why the user was not able to exercise in the second time period in a manner that satisfies the condition. For example, the control unit 101 displays on the display device 106 an inquiry screen that inquires as to the reason why the user could not exercise in the second time period in a manner that satisfies the condition.

In step S17, the control unit 101 operates as the information presentation unit 158 and obtains the answer input by the user using the input device 107, and determines whether or not there is a reason why the user could not exercise. When the user answers that there is no reason, then processing proceeds to step S20, and when the user has input a reason as an answer, the processing proceeds to step S18.

In step S18, the control unit 101 operates as the information presentation unit 158 and determines whether or not to accept the reason input by the user. When the control unit 101 determines that the reason input by the user is tolerant, the processing proceeds to step S19, otherwise the processing proceeds to step S20. The control unit 101 accepts the reason input by the user when the reason input by the user is reasonable such as work was busy. On the other hand, if the reason input by the user is not reasonable, the control unit 101 does not accept the reason input by the user.

In step S19, the control unit 101 operates as the information presentation unit 158 and presents advice that facilitates achieving the goal, to the user. For example, the control unit 101 displays on the display device 106 a message saying “Please keep in mind to exercise as much as you are able to. When you can't exercise, please take extra care not to eat as much as usual”.

In step S20, the control unit 101 operates as the information presentation unit 158 and presents advice that facilitates achieving the goal, to the user. For example, the control unit 101 displays on the display device 106 a message strongly encouraging exercise saying “You are not getting enough exercise. Be sure to exercise everyday to reduce your weight”.

In this manner, the terminal device 100 presents the user with advice tailored to the user's situation when the weight has not changed toward achieving the goal.

Furthermore, the processing procedure illustrated in FIG. 4 is merely an example and the processing procedure can be appropriately changed.

### Effects

The terminal device 100 according to the present embodiment asks the user as to the reason why the user could not perform exercise in the second time period, when the terminal device 100 determines that the weight has not changed in the first time period toward achieving the predetermined goal and the terminal device 100 determines that the user has not performed exercise in the second time period in a manner that satisfies the predetermined condition. The terminal device 100 presents to the user a message prompting to exercise as much as they are able to when the reason input by the user is tolerant, and presents to the user a message strongly prompting to exercise when the reason input by the user is not tolerant or the user answered that there is no reason. Accordingly, advice can be presented to the user without reducing the motivation of the user. In other words, appropriate advice that facilitates achieving a goal relating to health can be provided to the user.

The second time period may include at least a portion of the first time period and a day prior to the first time period. Weight in the initial portion of the first time period is affected by the exercise of the user in the time period prior to the first time period. By the second time period including the day prior to the first time period, whether or not the cause of the indicator not changing in the first time period toward achieving the predetermined goal is exercise can be determined with greater accuracy. In the case in which the first time period is a long time period, such as one month, the user's exercise in the time period prior to the first time period has little effect on the weight in the first time period. Thus, even when the second time period does not include the day prior to the first time period, whether or not the cause of the weight not changing in the first time period toward achieving the predetermined goal is exercise can be determined with accuracy. In the case in which the first time period is a short time period, such as one week, the second time period preferably includes the day prior to the first time period.

The terminal device 100 determines whether the first time period corresponds to one of the first stage in which weight has not reached the target value or the second stage in which weight has reached the target value, and determines whether or not weight has changed in the first time period toward achieving the goal according to different criteria for the first stage and the second stage. For example, for the first stage, in the case in which the weight indicates sign of a decreasing trend, the terminal device 100 determines that the weight has changed in the first time period toward achieving the goal and, in the case in which the weight does not indicate sign of a decreasing trend, the terminal device 100 determines that the weight has not changed in the first time period toward achieving the goal. For example, for the second stage, in the case in which the average value of the weight for the first time period is less than the target value, the terminal device 100 determines that the weight has changed in the first time period toward achieving the goal, and in the case in which the average value of the weight for the first time period is greater than the target value, the terminal device 100 determines that the weight has not changed in the first time period toward achieving the goal. Accordingly, the technology for managing health described above can be applied not only at the stage of reducing weight, but also at the stage of maintaining weight.

### Modified Examples

Note that the present invention is not limited to the embodiments described above.

The indicator relating to the user is information relating to the biological information or behavior of the user that is measurable by a sensor. Instead of weight, this may be heart rate, pulse rate, an electrocardiogram, pulse wave, hours of sleep, and the like. Instead of exercise, the user's behavior may be food intake, use of medication, drinking alcohol, sleep, work, and the like. The behavior may be able to be measured by a discretionary sensor or may be non-measurable. In an embodiment, the indicator associated with the user's biological information is blood pressure, and the behavior is the use of a medication (specifically, taking an antihypertensive agent).

In the above-described embodiments, the terminal device functioning as a health management device is a separate device from the measurement device (for example, a blood pressure monitor) that measures an indicator relating to a user. However, the health management device can be provided in the measurement device.

In short, the present invention is not limited to the embodiment described above as is, and the components can be modified and embodied within a range that does not depart from the gist in a stage of implementation. Further, various inventions can be formed by appropriately combining a plurality of constituent elements disclosed in the embodiment described above. For example, some constituent elements may be omitted from the entire constituent elements shown in the embodiment. Furthermore, the constituent elements of different embodiments may be combined appropriately.

### Supplementary Notes

A part or the entirety of the embodiment can be described, as described in the following supplementary notes in addition to the scope of the claims, but the present invention is not limited thereto.

### (Supplementary Note 1)

A health management device (20), including, a measurement data acquisition unit (21) configured to acquire measurement data output from a measurement device configured to measure an indicator relating to a user, a first determination unit (22) configured to determine, on the basis of the measurement data, whether or not the indicator has changed in a first time period toward achieving a preset goal, a behavior data acquisition unit (23) configured to acquire behavior data relating to behavior of the user, a second determination unit (24) configured to determine, on the basis of the behavior data, whether or not the user has performed the behavior in a second time period in a manner that satisfies a preset condition, the second time period including at least a portion of the first time period, an inquiry unit (25) configured to inquire the user as to a reason why the user could not perform the behavior in the second time period in a manner that satisfies the condition, in a case in which the first determination unit determines that the indicator has not changed in the first time period toward achieving the goal and the second determination unit determines that the user has not performed the behavior in the second time period in a manner that satisfies the condition, and an information presentation unit (26) configured to present, on the basis of an answer input by the user to an inquiry from the inquiry unit (25), information that facilitates achieving the goal, to the user.

### REFERENCE SIGNS LIST

10 Health management system
11 Weight scale
12 Exercise amount meter
20 Terminal device
21 Measurement data acquisition unit
22 First determination unit
23 Behavior data acquisition unit
24 Second determination unit
25 Inquiry unit
26 Information presentation unit
100 Terminal device
101 Control unit
102 CPU
103 RAM
104 ROM
105 Storage unit
106 Display device
107 Input device
108 Acceleration sensor
109 Speaker
110 Microphone
111 Communication interface
112 Battery
151 Input unit
152 Display control unit
153 Measurement data acquisition unit
154 First determination unit
155 Behavior data acquisition unit
156 Second determination unit
157 Inquiry unit
158 Information presentation unit
161 Measurement data storage unit
162 Behavior data storage unit
163 Database

1. A health management device, comprising:
one or more processors configured to:
acquire measurement data output from a measurement device configured to measure an indicator relating to a user;
determine, on the basis of the measurement data, whether or not the indicator has changed in a first time period toward achieving a preset goal;
acquire behavior data relating to behavior of the user;
determine, on the basis of the behavior data, whether or not the user has performed the behavior in a second time period in a manner that satisfies a preset condition, the second time period including at least a portion of the first time period;
inquire the user as to a reason why the user could not perform the behavior in the second time period in a manner that satisfies the condition, in a case in which it is determined that the indicator has not changed in the first time period toward achieving the goal and it is determined that the user has not performed the behavior in the second time period in a manner that satisfies the condition; and
present, on the basis of an answer input by the user to an inquiry, information that facilitates achieving the goal, to the user and to determine, in a case in which the answer includes a reason why the behavior could not be performed, whether or not to accept the reason included in the answer, and present different information to the user for a case in which the reason included in the answer is tolerant and for a case in which the reason included in the answer is not tolerant.

2. A health management device, comprising:
one or more processors configured to:
acquire measurement data output from a measurement device configured to measure an indicator relating to a user;
determine, on the basis of the measurement data, whether or not the indicator has changed in a first time period toward achieving a preset goal;
acquire behavior data relating to behavior of the user;
determine, on the basis of the behavior data, whether or not the user has performed the behavior in a second time period in a manner that satisfies a preset condition, the second time period including at least a portion of the first time period;
inquire the user as to a reason why the user could not perform the behavior in the second time period in a manner that satisfies the condition, in a case in which it is determined that the indicator has not changed in the first time period toward achieving the goal and it is determined that the user has not performed the behavior in the second time period in a manner that satisfies the condition; and
present, on the basis of an answer input by the user to an inquiry, information that facilitates achieving the goal, to the user, wherein
the goal includes a target value, and
the one or more processors are further configured to
determine, on the basis of the measurement data, whether the first time period corresponds to one of a first stage in which the indicator has not achieved the target value or a second stage in which the indicator has achieved the target value, and
determine, according to different criteria for the first stage and the second stage, whether or not the indicator has changed in the first time period toward achieving the goal.

3. The health management device according to claim 2, wherein
the one or more processors are further configured,
for the first stage, to determine that the indicator has changed in the first time period toward achieving the goal in a case in which the indicator shows sign of a decreasing trend, and determine that the indicator has not changed in the first time period toward achieving the goal in a case in which the indicator shows no sign of a decreasing trend, and
for the second stage, to determine that the indicator has changed in the first time period toward achieving the goal in a case in which an average value of the indicator for the first time period is less than the target value, and determine that the indicator has not changed in the first time period toward achieving the goal in a case in which the average value of the indicator for the first time period is greater than the target value.

4. The health management device according to claim 1, wherein
the second time period includes at least a portion of the first time period and a day prior to the first time period.

5. The health management device according to claim 2, wherein
the second time period includes at least a portion of the first time period and a day prior to the first time period.

6. A health management method executed by a health management device, the method comprising:
acquiring measurement data output from a measurement device configured to measure an indicator relating to a user;
determining, on the basis of the measurement data, whether or not the indicator has changed in a first time period toward achieving a preset goal;
acquiring behavior data relating to behavior of the user;
determining, on the basis of the behavior data, whether or not the user has performed the behavior in a second time period in a manner that satisfies a preset condition, the second time period including at least a portion of the first time period;
inquiring the user as to a reason why the user could not perform the behavior in the second time period in a manner that satisfies the condition, in a case in which it is determined that the indicator has not changed in the first time period toward achieving the goal and it is determined that the user has not performed the behavior in the second time period in a manner that satisfies the condition; and
presenting, on the basis of an answer input by the user to an inquiry as to the reason, information that facilitates achieving the goal, to the user, wherein
the presenting information that facilitates achieving the goal to the user includes, determining, in a case in which the answer includes a reason why the behavior could not be performed, whether or not to accept the reason included in the answer, and presenting different information to the user for a case in which the reason included in the answer is tolerant and for a case in which the reason included in the answer is not tolerant.

7. A non-transitory recording medium storing a program for causing a computer to perform functions in the health management device according to claim 1.

8. A non-transitory recording medium storing a program for causing a computer to perform functions in the health management device according to claim 2.

9. A health management method executed by a health management device, the method comprising:
acquiring measurement data output from a measurement device configured to measure an indicator relating to a user;
determining, on the basis of the measurement data, whether or not the indicator has changed in a first time period toward achieving a preset goal;
acquiring behavior data relating to behavior of the user;
determining, on the basis of the behavior data, whether or not the user has performed the behavior in a second time period in a manner that satisfies a preset condition, the second time period including at least a portion of the first time period;
inquiring the user as to a reason why the user could not perform the behavior in the second time period in a manner that satisfies the condition, in a case in which it is determined that the indicator has not changed in the first time period toward achieving the goal and it is determined that the user has not performed the behavior in the second time period in a manner that satisfies the condition; and
presenting, on the basis of an answer input by the user to an inquiry as to the reason, information that facilitates achieving the goal, to the user, wherein
the goal includes a target value, and
the determining whether or not the indicator has changed in a first time period toward achieving a preset goal includes
determining, on the basis of the measurement data, whether the first time period corresponds to one of a first stage in which the indicator has not achieved the target value or a second stage in which the indicator has achieved the target value, and
determining, according to different criteria for the first stage and the second stage, whether or not the indicator has changed in the first time period toward achieving the goal.
